(19) United States
(12) Patent Application Publication
(10) Pub. No.: US 2025/0363799 A1
(43) Pub. Date: Nov. 27, 2025

(54) CONTACTLESS PHYSIOLOGICAL MEASUREMENT SYSTEM HAVING ERROR COMPENSATION FUNCTION

(71) Applicant: FaceHeart Inc., Hsinchu City (TW)

(72) Inventors: Yi-Chiao Wu, New Taipei City (TW); Li-Wen Chiu, Tainan City (TW); Meng-Liang Chung, Changhua County (TW); Bing-Fei Wu, Hsinchu City (TW)

(73) Assignee: FaceHeart Inc., Hsinchu City (TW)

(21) Appl. No.: 19/295,820

(22) Filed: Aug. 11, 2025

Related U.S. Application Data
(63) Continuation-in-part of application No. 18/089,729, filed on Dec. 28, 2022.

(51) Int. Cl. G06V 10/98; A61B 5/00; A61B 5/0205; G06T 7/00; G06V 10/774; G06V 10/776; G06V 40/16

(52) U.S. Cl. CPC G06V 10/993; A61B 5/0205; A61B 5/7264; G06T 7/0012; G06V 10/774; G06V 10/776; G06V 40/161; G06T 2207/20081; G06T 2207/30168; G06T 2207/30201

(57) ABSTRACT

A contactless physiological measurement system having error compensation function is disclosed. The contactless physiological measurement system comprises a camera and an electronic device. According to the design of the present invention, the electronic device controls the camera to capture a user image and, after detecting a facial region from the user image, extracts an rPPG signal from the facial region. The electronic device then inputs the rPPG signal into a pre-trained physiological parameter estimation model to generate a preliminary physiological parameter. Specifically, the electronic device extracts at least one error-related feature from the facial region and inputs the error-related feature into a pre-trained error compensation parameter estimation model to generate an error compensation parameter. Consequently, a physiological parameter is produced by performing an addition operation between the error compensation parameter and the preliminary physiological parameter.

### CROSS-REFERENCE TO RELATED APPLICATIONS

This application is a Continuation-In-Part (CIP) of U.S. patent application Ser. No. 18/089,729, filed on Dec. 28, 2022, which is incorporated herein by reference in its entirety.

### BACKGROUND OF THE INVENTION

### 1. Field of the Invention

The present invention relates to a physiological signal measurement system, and more particularly to a contactless physiological measurement system having error compensation function for improving the accuracy of estimated physiological parameters.

### 2. Description of the Prior Art

The human face serves as an important source of information regarding a person's physical condition. For example, an individual may appear visibly pale or fatigued when experiencing illness. Consequently, monitoring physiological information plays a critical role in health assessment. Access to such physiological data is not only essential in clinical environments but is also increasingly important in various other domains, such as telemedicine, personal fitness, e-commerce, financial trading, and the management of mental stress induced by human-computer interaction.

In response to this need, an optical measurement technique known as photoplethysmography (PPG) has been developed and employed for the estimation of physiological parameters such as pulse and heart rate (HR). Referring to FIG. 1, a schematic perspective view of a conventional contactless physiological measurement device utilizing PPG technology is illustrated. FIG. 2 shows a block diagram of the same device. As shown in FIGS. 1 and 2, a conventional contactless physiological measurement device 1a primarily comprises a camera 11a and an electronic device 12a coupled thereto. The electronic device 12a includes a microprocessor 121a and a memory 122a coupled to the microprocessor 121a. The memory 122a stores a face detection program 123a and a physiological parameter estimation program 124a. When the face detection program 123a is executed, the microprocessor 121a is configured to detect a facial region (i.e., a region of interest or ROI) from an image captured by the camera 11a. Upon execution of the physiological parameter estimation program 124a, the microprocessor 121a extracts a remote photoplethysmograph (rPPG) signal from the detected facial region and applies at least one signal processing algorithm to estimate at least one physiological parameter, such as heart rate or pulse.

However, in real-world applications, it has been observed that artifacts resulting from motion and/or illumination variation can significantly degrade the accuracy of physiological parameters measured by the contactless physiological measurement device la. Various motion compensation techniques have been proposed to address this issue, along with improved versions of the physiological parameter estimation program. While these approaches offer partial improvements, they remain insufficient in ensuring reliable accuracy, especially under conditions with severe artifact interference.

In light of the foregoing, it is apparent that conventional contactless physiological measurement devices, including the face detection program 123a and the physiological parameter estimation program 124a, still leave room for improvement. Accordingly, the inventors of the present application have made diligent research efforts and have developed a contactless physiological measurement system and method equipped with an error compensation function to overcome these limitations.

### SUMMARY OF THE INVENTION

In order to solve the technical problems in the conventional technologies described above, the primary objective of the present invention is to provide a contactless physiological measurement system with error compensation function, which mainly comprises a camera and an electronic device. According to the design of the present invention, the electronic device controls the camera to capture a user image and, after detecting a facial region from the user image, extracts an rPPG signal from the facial region. The electronic device then inputs the rPPG signal into a pre-trained physiological parameter estimation model to generate a preliminary physiological parameter. Specifically, the electronic device extracts at least one error-related feature from the facial region and inputs the error-related feature into a pre-trained error compensation parameter estimation model to generate an error compensation parameter. Finally, a physiological parameter is produced by performing an addition operation between the error compensation parameter and the preliminary physiological parameter.

In brief, the contactless physiological measurement system with error compensation function according to the present invention is capable of eliminating the adverse effects caused by motion-and illumination-induced artifacts on the accuracy of physiological parameter measurement. In other words, even if the user is not in a stationary state or the ambient lighting conditions are unstable, the system of the present invention can still accurately measure the user's physiological parameters.

In order to achieve the aforementioned objective, one embodiment of the contactless physiological measurement system with error compensation function is provided in this disclosure, which comprises:
a camera, being configured to face a user; and
an electronic device, being coupled to or integrated with the camera, and further comprising a processor and a memory;
wherein the memory stores an application program, a pre-trained physiological parameter estimation model, and a pre-trained error parameter estimation model;
wherein the processor executes the application program and is thereby configured to:
acquire, by controlling the camera, a user image from the user;
detect, a facial region from the user image;
extract, a remote photoplethysmograph (rPPG) signal from the facial region;
input, the rPPG signal into the pre-trained physiological parameter estimation model, thereby generating a preliminary physiological parameter;
transform, the rPPG signal into a frequency-domain rPPG signal comprising K discrete frequencies;
obtain, by processing the facial region and the frequency-domain rPPG signal, a facial quality indices feature (f_{FQI});
obtain, by processing the frequency-domain rPPG signal, L detected frequencies within a specific frequency range for forming a frequency magnitude spectra feature (f_{MS});
generate, by inputting the facial quality indices feature and the frequency magnitude spectra feature into the pre-trained error parameter estimation model, an error compensation parameter; and
generate, by performing an addition operation between the preliminary physiological parameter and the error compensation parameter, a physiological parameter.

In one embodiment, the physiological parameter is selected from a group consisting of blood pressure, heart rate (HR), heart rate variance (HRV), blood oxygen saturation, pulse, and respiratory rate.

In one embodiment, the pre-trained error parameter estimation model is obtained through the following machine learning training process:
providing a plurality of training samples, wherein each of the plurality of training samples comprises a reference facial quality indices feature, a reference frequency magnitude spectra feature, and a reference preliminary physiological parameter obtained by inputting a reference rPPG signal into the pre-trained physiological parameter estimation model;
inputting the reference facial quality indices feature, the reference frequency magnitude spectra feature, and the reference preliminary physiological parameter into a machine learning model, thereby generating a predicted error compensation parameter corresponding to the training sample;
calculating an actual error based on a difference between the reference preliminary physiological parameter and a corresponding reference ground-truth physiological parameter;
comparing the predicted error compensation parameter with the actual error to calculate a prediction accuracy of the model;
in a case that the prediction accuracy of the model does not reach a predetermined accuracy threshold, adjusting model parameters of the machine learning model and repeatedly performing the training process described above until the model converges; and
in a case that the prediction accuracy of the model reaches the predetermined accuracy threshold, defining the trained machine learning model as the pre-trained error parameter estimation model.

In one embodiment, the facial region includes M×N pixels, and the facial quality indices feature comprises an average luminance, an average blue chrominance component, and an average red chrominance component of the M×N pixels. The application program comprises a first algorithm configured to calculate the average luminance, the average blue chrominance component, and the average red chrominance component, and the first algorithm comprises the following four mathematical expressions:

$$[ \begin{matrix}Y \\ Cb \\ Cb\end{matrix} ] = [ \begin{matrix}0.2126 & 0.7152 & 0.0722 \\ - 0.1146 & - 0.3854 & 0.5 \\ 0.5 & - 0.4542 & - 0.458\end{matrix} ] [ \begin{matrix}R \\ G \\ B\end{matrix} ] \qquad ( 1 )$$

$${Y}_{avg} = \frac{1}{{N}_{P}} {\sum }_{i = 1}^{N} {Y}_{i} \qquad ( 2 )$$

$${Cb}_{avg} = \frac{1}{{N}_{P}} {\sum }_{i = 1}^{N} {Cb}_{i} \qquad ( 3 )$$

$${Cr}_{avg} = \frac{1}{{N}_{P}} {\sum }_{i = 1}^{N} {Cr}_{i} \qquad ( 4 )$$

wherein K, L, M, and N are both positive integers, and Np=M×N;
wherein Y, Cb, Cr, R, G, and B correspondingly denote a luminance, a blue chrominance component, a red chrominance component, a red subpixel grayscale, a green subpixel grayscale, and a blue subpixel grayscale of one of the M×N pixels;
wherein Yᵢ, Cbᵢ and Crᵢ correspondingly denote the luminance, the blue chrominance component, the red chrominance component of an i-th pixel among the M×N pixels;
wherein Y_{avg}, Cb_{avg} and Cr_{avg} correspondingly denote the average luminance, the average blue chrominance component, and the average red chrominance component.

In one embodiment, the facial quality indices feature further comprises a facial region area, a skin mask area, and a skin mask ratio, and the application program further comprises a second algorithm configured to calculate the facial region area, the skin mask area, and the skin mask ratio, of which the second algorithm includes the following three mathematical expressions:

$${ROI}_{area} = {N}_{P} = ( {x}_{2} - {x}_{1} + 1 ) \times ( {y}_{2} - {y}_{1} + 1 ) \qquad ( 4 )$$

$${Skin}_{area} = {N}_{S} {\vert}_{{M}_{i} = 1} \qquad ( 5 )$$

$${Skin}_{ratio} = \frac{{N}_{S} {\vert}_{{M}_{i} = 1}}{{N}_{P}} \qquad ( 6 )$$

wherein (x₁, y₁) and (x₂, y₂) represent a top-left corner and a bottom-right corner of the facial region, respectively, and Mᵢ denotes a binary mask parameter corresponding to the i-th pixel;
wherein in case that the Cbᵢ of the i-th pixel falls within a first range between 77 and 127 as well as the Crᵢ of the i-th pixel falls within a second range between 133 and 173, Mᵢ is set to 1; otherwise, Mᵢ is set to 0;
wherein in case that there are U of the M×N pixels satisfy Mᵢ=1, N_{S}|_{M}_{ᵢ}₌₁=U;
wherein U is an integer.

In one embodiment, the specific frequency range is defined by a lower

frequency bound and an upper frequency bound, wherein the lower frequency bound and the upper frequency bound respectively correspond to a lowest frequency and a highest frequency.

In one embodiment, the facial quality indices feature further comprises a signal-to-noise ratio, and the application program further comprises a third algorithm for calculating the signal-to-noise ratio; wherein the third algorithm comprises the following three mathematical expressions:

$$SNR ( dB ) = 10 {log}_{10} ( \frac{{P}_{signal}}{{P}_{noise}} ) \qquad ( 8 )$$

$${P}_{signal} = {\sum }_{{f}_{i} ϵ [ {f}_{min} , {f}_{max} ]} {\vert S ( {f}_{i} ) \vert}^{2} \qquad ( 9 )$$

$${P}_{noise} = {\sum }_{{f}_{i} ∉ [ {f}_{min} , {f}_{max} ]} {\vert s ( {f}_{i} ) \vert}^{2} \qquad ( 10 )$$

wherein P_{signal} and Pₙₒᵢₛₑ represent a signal power and a noise power of the frequency-domain rPPG signal, respectively;
wherein fₘᵢₙ and fₘₐₓ represent the lowest frequency and the highest frequency;
wherein |S(fᵢ)|² represents a power corresponding to an i-th detected frequency among the L detected frequencies.

In one embodiment, the application program further comprises a sorting algorithm, and the processor executes the sorting algorithm so as to be configured to:
sort, based on the power, the L detected frequencies so as to form the frequency magnitude spectra feature.

In one embodiment, in case that the electronic device includes the camera, the electronic device is selected from a group consisting of smartphone, tablet computer, smart television, video door phone, facial recognition attendance device, desktop computer, laptop computer, all-in-one computer, and in-vehicle infotainment (IVI) device.

In one embodiment, the camera is integrated into a user electronic device, such that the electronic device is coupled to the camera via the user electronic device. In one embodiment, the user electronic device is selected from a group

consisting of smartphone, tablet computer, smart television, video door phone, facial recognition attendance device, desktop computer, laptop computer, all-in-one computer, and in-vehicle infotainment (IVI) device.

### BRIEF DESCRIPTION OF THE DRAWINGS

The invention as well as a preferred mode of use and advantages thereof will be best understood by referring to the following detailed description of an illustrative embodiment in conjunction with the accompanying drawings, wherein:

FIG. 1 is a perspective view of a conventional contactless physiological measurement device.

FIG. 2 is a block diagram of the conventional contactless physiological measurement device.

FIG. 3 is a first structural diagram of a contactless physiological measurement system having error compensation function according to the present invention.

FIG. 4 is a second structural diagram of the contactless physiological measurement system having error compensation function according to the present invention.

FIG. 5 is a block diagram of the contactless physiological measurement system having error compensation function as shown in FIG. 3.

FIG. 6 is a spectral diagram of a frequency-domain rPPG signal.

FIG. 7 is a bar chart showing bin indices versus bin serial numbers.

FIG. 8 is a bar chart showing normalized bin indices versus bin serial numbers.

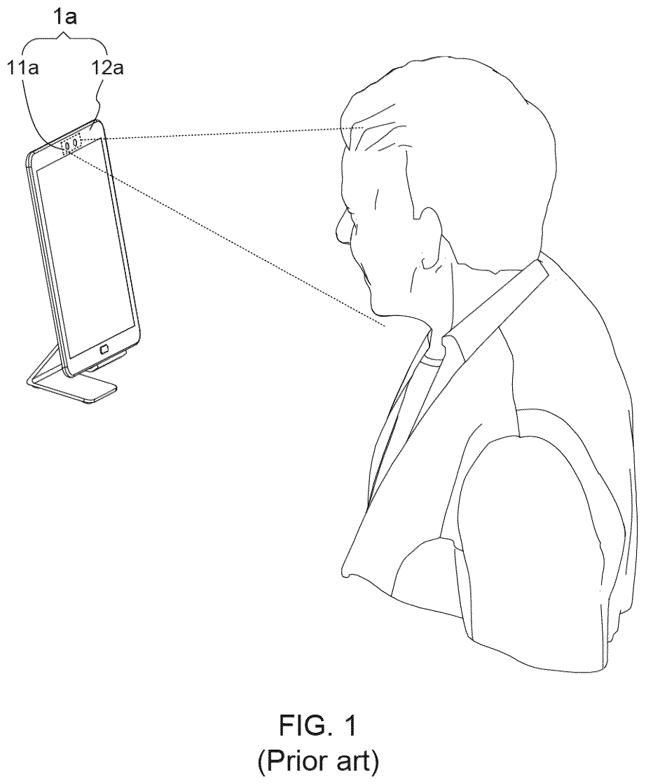

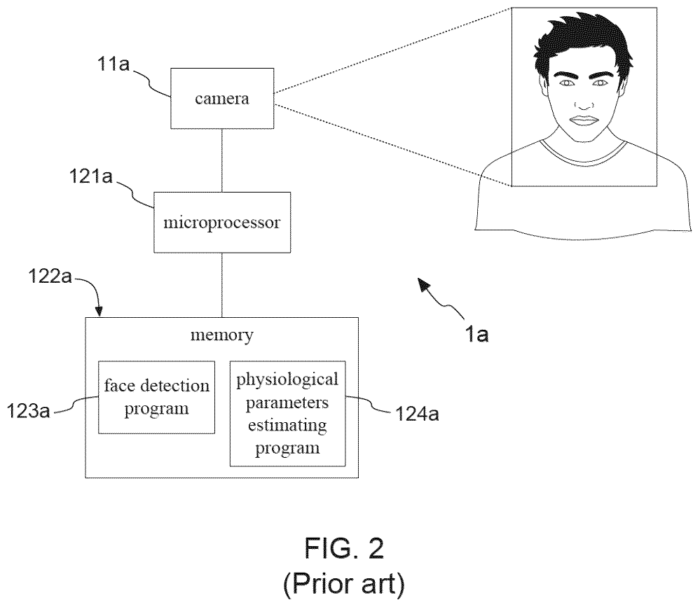

### DETAILED DESCRIPTION OF THE PREFERRED EMBODIMENTS

The objectives, technical contents and features of this disclosure will become apparent in the following detailed description of the preferred embodiments with reference to the accompanying drawings. It is noteworthy that the drawings used in the specification and subject matters of this disclosure are intended for illustrating the technical characteristics of this disclosure, but not necessarily to be drawn according to actual proportion and precise configuration. Therefore, the scope of this disclosure should not be limited to the proportion and configuration of the drawings.

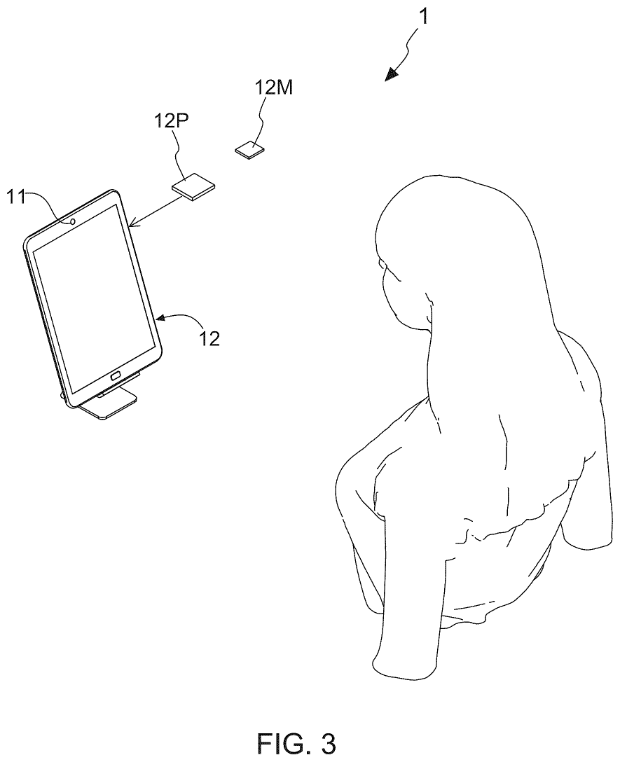

With reference to FIG. 3, a first structural diagram of a contactless physiological measurement system having error compensation function according to the present invention is illustrated. As shown in FIG. 3, the contactless physiological measurement system 1 comprises an electronic device 12 and a camera 11 that is configured to face a user, of which the camera 11 is integrated into the electronic device 12, and the electronic device 12 comprises a processor 12P and a memory 12M. In feasible embodiments, the electronic device 12 may be, but is not limited to, a smartphone, a tablet computer, a smart television, a video door phone, a facial recognition attendance device, a desktop computer, a laptop computer, an all-in-one computer, or an in-vehicle infotainment device.

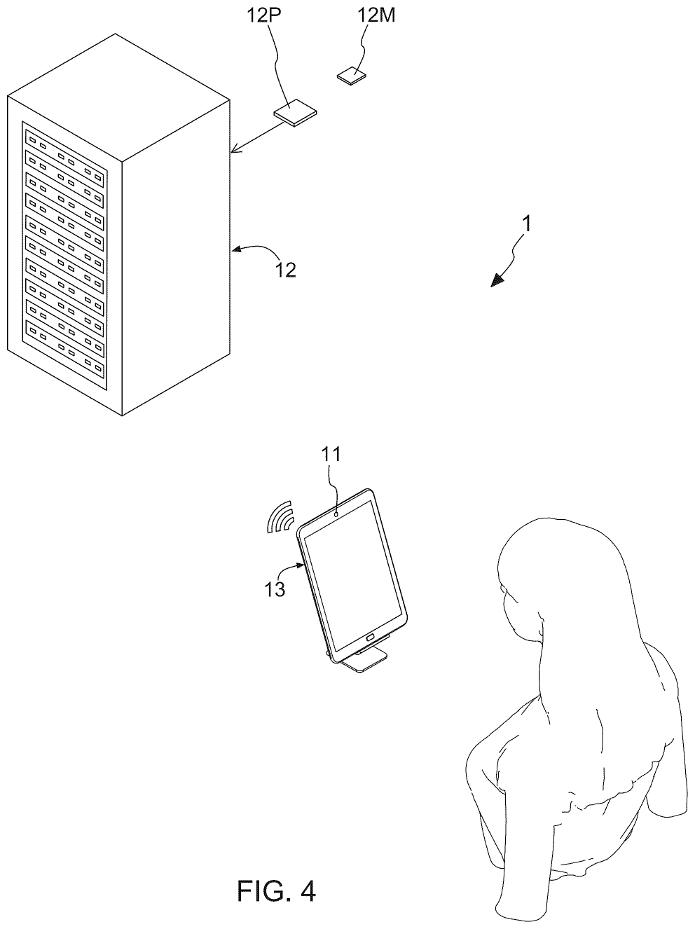

On the other hand, FIG. 4 illustrates a second structural diagram of the contactless physiological measurement system having error compensation function according to the present invention. As shown in FIG. 4, the contactless physiological measurement system 1 comprises a camera 11 and an electronic device 12, wherein the camera 11 is integrated into a user electronic device 13, and the electronic device 12 comprises a processor 12P and a memory 12M. With such design, the camera 11 is coupled to the electronic device 12 through the user electronic device 13. In feasible embodiments, the electronic device 12 may be a remote server, a local server, or an edge server. In addition, the user electronic device 13 may be, but is not limited to, a smartphone, a tablet computer, a smart television, a video door phone, a facial recognition attendance device, a desktop computer, a laptop computer, an all-in-one computer, or an in-vehicle infotainment device.

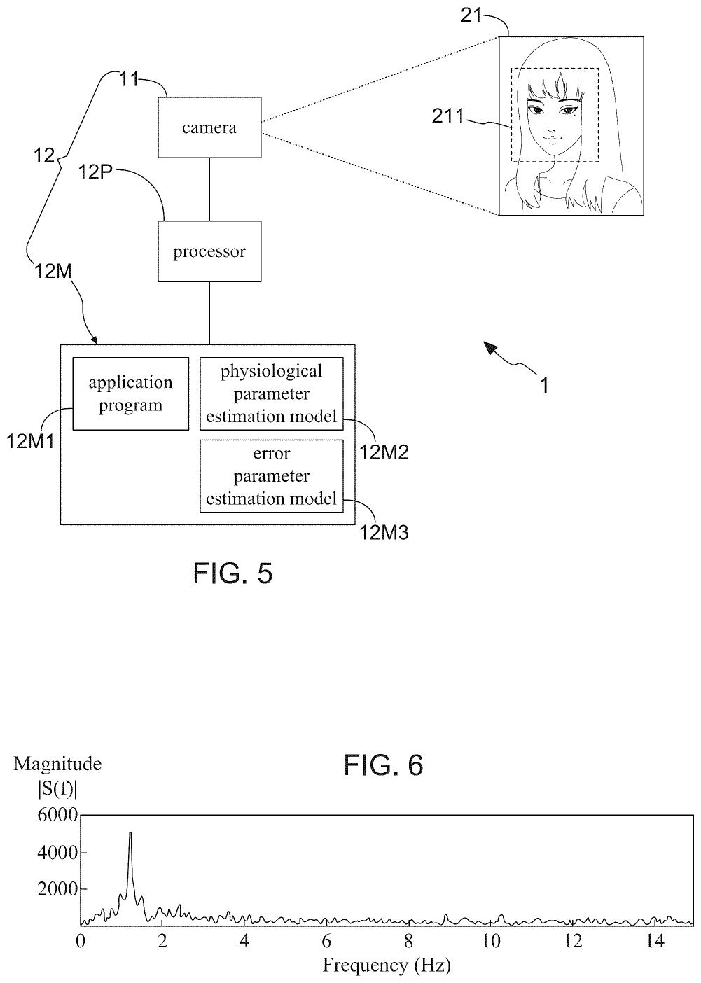

Furthermore, FIG. 5 depicts a block diagram of the contactless physiological measurement system in FIG. 3. As FIG. 3 and FIG. 5 show, in the electronic device 12 the memory 12M stores an application program 12M1, a pre-trained physiological parameter estimation model 12M2, and a pre-trained error parameter estimation model 12M3. More specifically, the application program 12M1 comprises a main control module, a plurality of function modules, and a plurality of algorithms. The main control module is configured to manage the execution flow of each of the function modules, and at corresponding timing points, import data to be processed or already processed into a corresponding algorithm or input the data into a corresponding estimation model to perform a specific computation. Therefore, when the application program 12M1 is executed, the processor 12P, by executing the application program 12M1, is configured to:
control the camera 11 to acquire a user image 21 from the user;
detect a facial region 211 from the user image 21;
extract a remote photoplethysmograp (rPPG) signal from the facial region 211, and input the rPPG signal into the pre-trained physiological parameter estimation model 12M2 to generate a preliminary physiological parameter;
transform the rPPG signal into a frequency-domain rPPG signal comprising K discrete frequencies;
process the facial region 211 and the frequency-domain rPPG signal to obtain a facial quality indices feature (f_{FQI});
process the frequency-domain rPPG signal to extract L detected frequencies within a specific frequency range, thereby forming a frequency magnitude spectra feature (f_{MS});
input the facial quality indices feature and the frequency magnitude spectra feature into the pre-trained error parameter estimation model 12M3 to generate an error compensation parameter; and
perform an addition operation between the preliminary physiological parameter and the error compensation parameter to generate a physiological parameter.

In an exemplary embodiment, the physiological parameter may be, but is not limited to, blood pressure, heart rate (HR), heart rate variance (HRV), blood oxygen saturation, pulse, or respiratory rate. Furthermore, the step of “detecting a facial region 211 from the user image 21” is performed by a face detection model, which is one of the plurality of function modules. In one embodiment, the face detection model may be a multi-task convolutional neural networks (MTCNN) model, or other known face detection models such as:
FaceBoxes: a single-stage convolutional neural network-based face detection model;
RetinaFace: a powerful face detection model with facial landmark localization capability;
BlazeFace: a lightweight face detection model proposed by Google for mobile devices;
OpenCV Haar Cascade: a face detection method based on haar-like features and adaboost classifier;
Dlib CNN face detector: a Dlib face detection algorithm based on deep convolutional neural networks; or
YOLO-face: a face detection model based on the YOLO object detection architecture.

It is worth mentioning that the pre-trained physiological parameter estimation model 12M2 is established through the following machine learning training process:
providing a plurality of training samples, wherein each of the plurality of training samples comprises a reference facial quality indices feature, a reference frequency magnitude spectra feature, and a reference preliminary physiological parameter obtained by inputting a reference rPPG signal into the pre-trained physiological parameter estimation model 12M2;
inputting the reference facial quality indices feature, the reference frequency magnitude spectra feature, and the reference preliminary physiological parameter into a machine learning model, thereby generating a predicted error compensation parameter corresponding to the training sample;
calculating an actual error based on a difference between the reference preliminary physiological parameter and a corresponding reference ground-truth physiological parameter;
comparing the predicted error compensation parameter with the actual error to calculate a prediction accuracy of the model;
in a case that the prediction accuracy of the model does not reach a predetermined accuracy threshold, adjusting model parameters of the machine learning model and repeatedly performing the training process described above until the model converges; and
in a case that the prediction accuracy of the model reaches the predetermined accuracy threshold, defining the trained machine learning model as the pre-trained error parameter estimation model 12M3.

The foregoing machine learning model may be a self-constructed neural network, or may refer to existing physiological parameter estimation models developed based on rPPG technology, such as rPPG-MAE, PaPaGei, or PhySU-Net. However, due to region-specific factors such as skin tone distribution among different ethnic groups, facial features, and ambient lighting conditions, models like rPPG-MAE, PaPaGei, or PhySU-Net cannot be directly adopted as the pre-trained physiological parameter estimation model 12M2 of the present system. Retraining or parameter fine-tuning based on regional characteristics is still required.

Specifically, this system constructs an error feature (f_{ERROR}) by concatenating the facial quality indices feature (f_{FQI}) and the frequency magnitude spectra feature (f_{MS}), and the error feature is represented as: f_{ERROR}=concat(f_{FQI}, f_{MS}). In one embodiment, the facial region 211 comprises M×N pixels, and the facial quality indices feature includes an average luminance (Y_{avg}), an average blue-difference chrominance (Cb_{avg}), and an average red-difference chrominance (Cr_{avg}) of the M×N pixels. Furthermore, the application program 12M1 includes a first algorithm for calculating the average luminance, the average blue-difference chrominance, and the average red-difference chrominance. The first algorithm includes the following four mathematical equations:

$$[ \begin{matrix}Y \\ Cb \\ Cb\end{matrix} ] = [ \begin{matrix}0.2126 & 0.7152 & 0.0722 \\ - 0.1146 & - 0.3854 & 0.5 \\ 0.5 & - 0.4542 & - 0.458\end{matrix} ] [ \begin{matrix}R \\ G \\ B\end{matrix} ] \qquad ( 1 )$$

$${Y}_{avg} = \frac{1}{{N}_{P}} {\sum }_{i = 1}^{N} {Y}_{i} \qquad ( 2 )$$

$${Cb}_{avg} = \frac{1}{{N}_{P}} {\sum }_{i = 1}^{N} {Cb}_{i} \qquad ( 3 )$$

$${Cr}_{avg} = \frac{1}{{N}_{P}} {\sum }_{i = 1}^{N} {Cr}_{i} \qquad ( 4 )$$

To be explained in more detail below, K, L, M, and N are all positive integers, and Nₚ=M×N. On the other hand, Y, Cb, Cr, R, G, and B correspondingly denote a luminance, a blue chrominance component, a red chrominance component, a red subpixel grayscale, a green subpixel grayscale, and a blue subpixel grayscale of one of the M×N pixels. Furthermore, Yᵢ, Cbᵢ and Crᵢ correspondingly denote the luminance, the blue chrominance component, the red chrominance component of an i-th pixel among the M×N pixels.

In a feasible embodiment, the facial quality indices feature further includes a facial region area (ROIₐᵣₑₐ), a skin mask area (Skinₐᵣₑₐ), and a skin mask ratio (Skinᵣₐₜᵢₒ). Correspondingly, the application program also includes a second algorithm for calculating the facial region area, the skin mask area, and the skin mask ratio. The second algorithm includes the following three mathematical equations:

$${ROI}_{area} = {N}_{P} = ( {x}_{2} - {x}_{1} + 1 ) \times ( {y}_{2} - {y}_{1} + 1 ) \qquad ( 4 )$$

$${Skin}_{area} = {N}_{S} {\vert}_{{M}_{i} = 1} \qquad ( 5 )$$

$${Skin}_{ratio} = \frac{{N}_{S} {\vert}_{{M}_{i} = 1}}{{N}_{P}} \qquad ( 6 )$$

In the foregoing three mathematical equations, (x₁, y₁) and (x₂, y₂) represent a top-left corner and a bottom-right corner of the facial region 211, respectively, and Mᵢ denotes a binary mask parameter corresponding to the i-th pixel. To be more specific, in case that the Cbᵢ of the i-th pixel falls within a first range between 77 and 127 as well as the Crᵢ of the i-th pixel falls within a second range between 133 and 173, Mᵢ is set to 1; otherwise, Mᵢ is set to 0. Furthermore, in case that there are U (i.e., an integer) of the M×N pixels satisfy Mᵢ=1, Nₛ|_{M}_{ᵢ}₌₁=U.

It is further explained that the specific frequency range is defined by a lower frequency bound and an upper frequency bound, wherein the lower frequency bound and the upper frequency bound respectively correspond to a lowest frequency (fₘᵢₙ) and a highest frequency (fₘₐₓ).

Furthermore, FIG. 6 illustrates a spectral diagram of the frequency-domain rPPG signal. In one embodiment, the facial quality indices feature further includes a signal-to-noise ratio (SNR(dB)). Correspondingly, the application program further comprises a third algorithm configured to compute the signal-to-noise ratio, and the third algorithm includes the following three mathematical equations:

$$SNR ( dB ) = 10 {log}_{10} ( \frac{{P}_{signal}}{{P}_{noise}} ) \qquad ( 8 )$$

$${P}_{signal} = {\sum }_{{f}_{i} ϵ [ {f}_{min} , {f}_{max} ]} {\vert S ( {f}_{i} ) \vert}^{2} \qquad ( 9 )$$

$${P}_{noise} = {\sum }_{{f}_{i} ∉ [ {f}_{min} , {f}_{max} ]} {\vert s ( {f}_{i} ) \vert}^{2} \qquad ( 10 )$$

In the foregoing three mathematical equations, P_{signal} and Pₙₒᵢₛₑ represent a signal power and a noise power of the frequency-domain rPPG signal, respectively. On the other hand, if |S(fᵢ)| denotes a magnitude of an i-th detected frequency among the L detected frequencies, then |s(fᵢ)|² indicates corresponding energy intensity (also referred to as power) of the i-th detected frequency. Thus, it is evident that f_{FQI} comprises multiple features selected from Y_{avg}, Cb_{avg}, Cr_{avg}, ROIₐᵣₑₐ, Skinₐᵣₑₐ, Skinᵣₐₜᵢₒ, and SNR(dB).

It is reiterated that, by processing the frequency-domain rPPG signal, L detected frequencies within a specific frequency range (i.e., 0.5-3.3 Hz) can be extracted to form said frequency magnitude spectra feature (f_{MS}), wherein K>L.

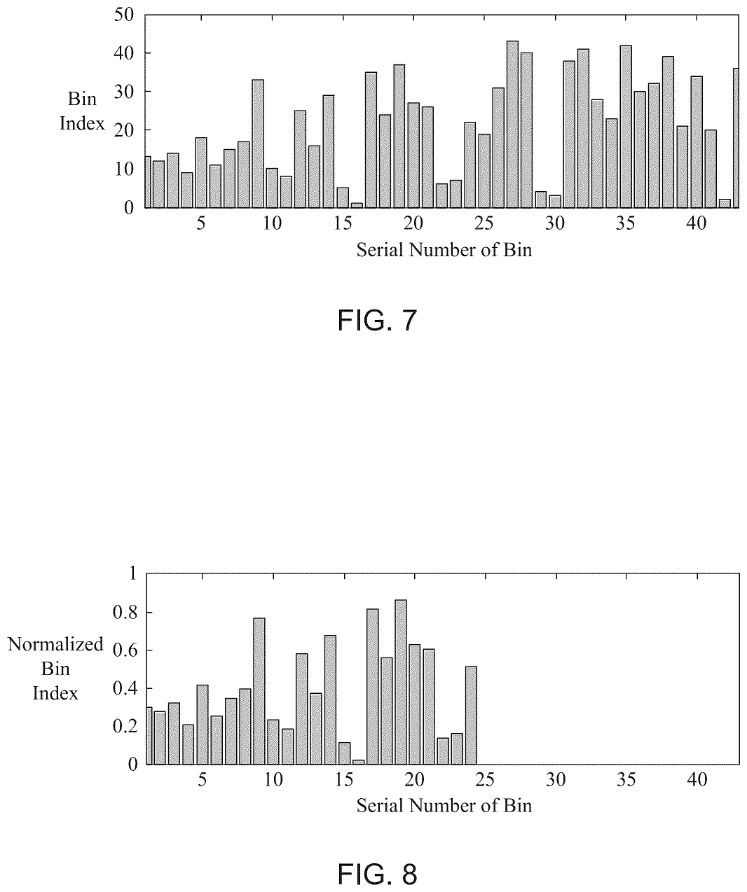

Specifically, assuming that the rPPG signal is subjected to a Fast Fourier Transform (FFT) with a total number of discrete frequency points N=300 and a sampling rate fs=30 Hz, then the frequency resolution of each frequency bin is fs/N=0.1 Hz. In further detail, the frequency-domain rPPG signal shown in FIG. 6 covers a frequency range from 0 Hz to 15 Hz, and therefore comprises 150 frequency bins in total. However, given that the effective frequency range related to heart rate (HR) lies between 0.5 Hz and 3.3 Hz, and in order to avoid noise interference and focus on the low-frequency range with higher physiological information density, as shown in FIG. 7, the processor 12P is configured to selects the first 45 frequency bins as candidates, namely those ranging from 0.1 Hz to 4.5 Hz. Subsequently, it is calculated that the bin index corresponding to 0.5 Hz is

$$\frac{0.5}{0.1} = 5 ,$$

and that corresponding to 3.3 Hz is

$$\frac{3.3}{0.1} = 33 .$$

Accordingly, 29 bins corresponding to the range between 0.5 Hz and 3.3 Hz are therefore selected from the 45 candidate bins. In other words, K=45 and L=29.

It is further noted that, in FIG. 7, an i-th bar represents an i-th frequency bin of the 45 candidate frequency bins. As explained in detail below, the Y-axis value indicates the bin index of the i-th frequency bin, where

$$bin index = \frac{{f}_{i}}{{f}_{s} / N} ,$$

and fᵢ is the discrete frequency corresponding to the i-th bin.

In addition, the application program further includes a sorting algorithm, and the processor 12P executes the sorting algorithm and is thereby configured to: sort the L detected frequencies according to their corresponding power values (i.e., |S(f_{bin=5})|², |S(f_{bin=6})|², . . . , |S(f_{bin=32})|², and |S(f_{bin=33})|²), so as to form said frequency magnitude spectra feature (f_{MS}). As a result, the 29 frequency bins after sorting are shown in FIG. 8.

After obtaining the facial quality indices feature (f_{FQI}) and the frequency magnitude spectra feature (f_{MS}), the processor 12P combines f_{FQI} and f_{MS} to form an error feature (f_{ERROR}=concat(f_{FQI}, f_{MS})), and then inputs the error feature into the pre-trained error parameter estimation model 12M3 to generate an error compensation parameter. Consequently, the processor 12P performs an addition operation between the preliminary physiological parameter and the error compensation parameter, thereby generating a physiological parameter. Specifically, the above two processing steps can be expressed by the following equations (11), (12), and (13):

$${f}_{ERROR} = concat ( {f}_{FQI} , {f}_{MS} ) \qquad ( 11 )$$

$$c = N ( {f}_{ERROR} ) \qquad ( 12 )$$

$$h = {h}^{′} + c = {h}^{′} + N ( {f}_{ERROR} ) \qquad ( 13 )$$

In the foregoing three equations, c denotes to the error compensation parameter, h′ is the preliminary physiological parameter, and h represents the physiological parameter. On the other hand, it is not difficult to understand that N (f_{ERROR}) denotes said pre-trained error parameter estimation model 12M3. In practice, the pre-trained error parameter estimation model 12M3 can be obtained through the following machine learning training process:
providing a plurality of training samples, wherein each of the training samples includes a reference facial quality indices feature, a reference frequency magnitude spectra feature, and a reference preliminary physiological parameter obtained by inputting a reference rPPG signal into the pre-trained physiological parameter estimation model 12M2;
inputting the reference facial quality indices feature, the reference frequency magnitude spectra feature, and the reference preliminary physiological parameter into a machine learning model to generate a predicted error compensation parameter corresponding to the training sample;
calculating an actual error based on a difference between the reference preliminary physiological parameter and a corresponding reference ground-truth physiological parameter;
comparing the predicted error compensation parameter with the actual error to calculate a prediction accuracy of the model;
if the prediction accuracy of the model does not reach a predetermined accuracy threshold, adjusting the model parameters of the machine learning model and repeating the training process described above until the model converges; and
when the prediction accuracy of the model reaches the predetermined accuracy threshold, defining the trained machine learning model as the pre-trained error parameter estimation model 12M3.

It is further noted that the aforementioned statement: “when the prediction accuracy of the model does not reach a predetermined accuracy threshold, adjusting the model parameters of the machine learning model” can be represented by the following equations (14) and (15):

$$l ( W , {( {f}_{ERROR} , {h}^{′} , \hat{h} )}^{2} ) = {‖ N ( {f}_{ERROR}^{i} , W ) + {h}^{′ i} - {\hat{h}}^{i} ‖}_{2}^{2} \qquad ( 14 )$$

$$L ( W ) = {\sum }_{i = 1}^{P} l ( W , {( {f}_{ERROR} , {h}^{′} , \hat{h} )}^{2} ) \qquad ( 15 )$$

In the foregoing two equations, P denotes a total number of training samples, W represents the weight to be adjusted, ĥ denotes a reference actual physiological parameter, l(·) indicates the loss of a single training sample, and L(W) represents the total loss over all training samples. It should be understood that ∥·∥² is the L2 norm (also known as the Euclidean distance), and ∥·∥₂² represents the squared L2 norm, i.e., the mean square error (MSE).

Thus, the present invention has been described thoroughly and clearly, particularly regarding a contactless physiological measurement system having error compensation function. In summary, the contactless physiological measurement system having error compensation function according to the present invention offers the following advantages: it can eliminate adverse effects caused by motion-induced or illumination-induced artifacts on the accuracy of physiological parameter measurement. In other words, even when the user is in a non-stationary state or when the ambient lighting is unstable, the proposed system can still accurately measure the user's physiological parameters.

The embodiments used to explain technical philosophy and characteristics in the present invention are aimed at the present disclosure understood and put into practice by persons skilled in the art but not considered as restrictions to claims hereinafter, that is, any equivalent change or modification based on spirit of the preset disclosure should be included in claims hereinafter. Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A contactless physiological measurement system having error compensation function, comprising:
a camera, being configured to face a user; and
an electronic device, being coupled to or integrated with the camera, and further comprising a processor and a memory;
wherein the memory stores an application program, a pre-trained physiological parameter estimation model, and a pre-trained error parameter estimation model;
wherein the processor executes the application program and is thereby configured to:
acquire, by controlling the camera, a user image from the user;
detect, a facial region from the user image;
extract, a remote photoplethysmograph (rPPG) signal from the facial region;
input, the rPPG signal into the pre-trained physiological parameter estimation model, thereby generating a preliminary physiological parameter;
transform, the rPPG signal into a frequency-domain rPPG signal comprising K discrete frequencies;
obtain, by processing the facial region and the frequency-domain rPPG signal, a facial quality indices feature (f_{FQI});
obtain, by processing the frequency-domain rPPG signal, L detected frequencies within a specific frequency range for forming a frequency magnitude spectra feature (f_{MS});
generate, by inputting the facial quality indices feature and the frequency magnitude spectra feature into the pre-trained error parameter estimation model, an error compensation parameter; and
generate, by performing an addition operation between the preliminary physiological parameter and the error compensation parameter, a physiological parameter.

2. The contactless physiological measurement system of claim 1, wherein the physiological parameter is selected from a group consisting of blood pressure, heart rate (HR), heart rate variance (HRV), blood oxygen saturation, pulse, and respiratory rate.

3. The contactless physiological measurement system of claim 1, wherein the pre- trained error parameter estimation model is obtained through the following machine learning training process:
providing a plurality of training samples, wherein each of the plurality of training samples comprises a reference facial quality indices feature, a reference frequency magnitude spectra feature, and a reference preliminary physiological parameter obtained by inputting a reference rPPG signal into the pre-trained physiological parameter estimation model;
inputting the reference facial quality indices feature, the reference frequency magnitude spectra feature, and the reference preliminary physiological parameter into a machine learning model, thereby generating a predicted error compensation parameter corresponding to the training sample;
calculating an actual error based on a difference between the reference preliminary physiological parameter and a corresponding reference ground-truth physiological parameter;
comparing the predicted error compensation parameter with the actual error to calculate a prediction accuracy of the model;
in a case that the prediction accuracy of the model does not reach a predetermined accuracy threshold, adjusting model parameters of the machine learning model and repeatedly performing the training process described above until the model converges; and
in a case that the prediction accuracy of the model reaches the predetermined accuracy threshold, defining the trained machine learning model as the pre-trained error parameter estimation model.

4. The contactless physiological measurement system of claim 1, wherein the facial region includes M×N pixels, and the facial quality indices feature comprises an average luminance, an average blue chrominance component, and an average red chrominance component of the M×N pixels. The application program comprises a first algorithm configured to calculate the average luminance, the average blue chrominance component, and the average red chrominance component, and the first algorithm comprises the following four mathematical expressions:
$$[ \begin{matrix}Y \\ Cb \\ Cb\end{matrix} ] = [ \begin{matrix}0.2126 & 0.7152 & 0.0722 \\ - 0.1146 & - 0.3854 & 0.5 \\ 0.5 & - 0.4542 & - 0.458\end{matrix} ] [ \begin{matrix}R \\ G \\ B\end{matrix} ] ; \qquad ( 1 )$$
$${Y}_{avg} = \frac{1}{{N}_{P}} {\sum }_{i = 1}^{N} {Y}_{i} ; \qquad ( 2 )$$
$${Cb}_{avg} = \frac{1}{{N}_{P}} {\sum }_{i = 1}^{N} {Cb}_{i} ; \qquad ( 3 )$$
$${Cr}_{avg} = \frac{1}{{N}_{P}} {\sum }_{i = 1}^{N} {Cr}_{i} ; \qquad ( 4 )$$
wherein K, L, M, and N are all positive integers, and N_{P}=M×N;
wherein Y, Cb, Cr, R, G, and B correspondingly denote a luminance, a blue chrominance component, a red chrominance component, a red subpixel grayscale, a green subpixel grayscale, and a blue subpixel grayscale of one of the M×N pixels;
wherein Yᵢ, Cbᵢ and Crᵢ correspondingly denote the luminance, the blue chrominance component, the red chrominance component of an i-th pixel among the M×N pixels;
wherein Y_{avg}, Cb_{avg} and Cr_{avg} correspondingly denote the average luminance, the average blue chrominance component, and the average red chrominance component.

5. The contactless physiological measurement system of claim 4, wherein the facial quality indices feature further comprises a facial region area, a skin mask area, and a skin mask ratio, and the application program further comprises a second algorithm configured to calculate the facial region area, the skin mask area, and the skin mask ratio, of which the second algorithm includes the following three mathematical expressions:
$${ROI}_{area} = {N}_{P} = ( {x}_{2} - {x}_{1} + 1 ) \times ( {y}_{2} - {y}_{1} + 1 ) ; \qquad ( 4 )$$
$${Skin}_{area} = {N}_{S} {\vert}_{{M}_{i} = 1} ; \qquad ( 5 )$$
$${Skin}_{ratio} = \frac{{N}_{S} {\vert}_{{M}_{i} = 1}}{{N}_{P}} ; \qquad ( 6 )$$
wherein (x₁, y₁) and (x₂, y₂) represent a top-left corner and a bottom-right corner of the facial region, respectively, and Mᵢ denotes a binary mask parameter corresponding to the i-th pixel;
wherein in case that the Cbᵢ of the i-th pixel falls within a first range between 77 and 127 as well as the Crᵢ of the i-th pixel falls within a second range between 133 and 173, Mᵢ is set to 1; otherwise, Mᵢ is set to 0;
wherein in case that there are U of the M×N pixels satisfy Mᵢ=1, N_{S}|_{M}_{ᵢ}₌₁=U;
wherein U is an integer.

6. The contactless physiological measurement system of claim 5, wherein the specific frequency range is defined by a lower frequency bound and an upper frequency bound, wherein the lower frequency bound and the upper frequency bound respectively correspond to a lowest frequency and a highest frequency.

7. The contactless physiological measurement system of claim 6, wherein the facial quality indices feature further comprises a signal-to-noise ratio, and the application program further comprises a third algorithm for calculating the signal-to-noise ratio; wherein the third algorithm comprises the following three mathematical expressions:
$$SNR ( dB ) = 10 {log}_{10} ( \frac{{P}_{signal}}{{P}_{noise}} ) ; \qquad ( 8 )$$
$${P}_{signal} = {\sum }_{{f}_{i} ϵ [ {f}_{min} , {f}_{max} ]} {\vert S ( {f}_{i} ) \vert}^{2} ; \qquad ( 9 )$$
$${P}_{noise} = {\sum }_{{f}_{i} ∉ [ {f}_{min} , {f}_{max} ]} {\vert s ( {f}_{i} ) \vert}^{2} ; \qquad ( 10 )$$
wherein P_{signal} and Pₙₒᵢₛₑ represent a signal power and a noise power of the frequency-domain rPPG signal, respectively;
wherein fₘᵢₙ and fₘₐₓ represent the lowest frequency and the highest frequency;
wherein |S(fᵢ)|² represents a power corresponding to an i-th detected frequency among the L detected frequencies.

8. The contactless physiological measurement system of claim 6, wherein the application program further comprises a sorting algorithm, and the processor executes the sorting algorithm so as to be configured to:
sort, based on the power, the L detected frequencies so as to form the frequency magnitude spectra feature.

9. The contactless physiological measurement system of claim 1, wherein in case that the electronic device includes the camera, the electronic device is selected from a group consisting of smartphone, tablet computer, smart television, video door phone, facial recognition attendance device, desktop computer, laptop computer, all-in-one computer, and in-vehicle infotainment (IVI) device.

10. The contactless physiological measurement system of claim 1, wherein the camera is integrated into a user electronic device, such that the electronic device is coupled to the camera via the user electronic device.

11. The contactless physiological measurement system of claim 10, wherein the user electronic device is selected from a group consisting of smartphone, tablet computer, smart television, video door phone, facial recognition attendance device, desktop computer, laptop computer, all-in-one computer, and in-vehicle infotainment (IVI) device.
